(19) United States
(12) Patent Application Publication
(10) Pub. No.: US 2018/0035874 A1
(43) Pub. Date: Feb. 8, 2018

(54) APPARATUS AND METHOD FOR FIXATION TO ENDOSCOPE ACCESSORIES

(71) Applicant: The General Hospital Corporation, Boston, MA (US)

(72) Inventors: GUILLERMO J. TEARNEY, Cambridge, MA (US); TIMOTHY N. FORD, Somerville, MA (US); MICHAEL CALHOUN, Lighthouse Point, FL (US); ROBERT CARRUTH, Arlington, MA (US)

(21) Appl. No.: 15/550,669

(22) PCT Filed: Feb. 10, 2016
(86) PCT No.: PCT/US16/17372

§ 371 Date: Aug. 11, 2017

Related U.S. Application Data
(60) Provisional application No. 62/115,190, filed on Feb. 12, 2015.

(51) Int. Cl. A61B 1/00; A61B 1/012

(52) U.S. Cl. CPC A61B 1/00135; A61B 1/0125; A61B 1/00174

(57) ABSTRACT

A probe coupling apparatus can be provided which can include, e.g., at least two opposing edges, one of the edges being configured to receive a tip of a probe. The exemplary apparatus can also include an orifice provided between the edges and a portion provided between the opposing edges and extending along the orifice. The portion can have a structure such that, when a force is applied to a section of the portion, a cross-sectional area of the orifice is increase, and, when the force is released, the area is decreased.

### CROSS-REFERENCE TO RELATED APPLICATION(S)

This application is based upon and claims the benefit of priority from U.S. Patent Application Ser. No. 62/115,190, filed on Feb. 12, 2015, the entire disclosure of which is incorporated herein by reference.

### FIELD OF THE DISCLOSURE

The present disclosure relates to exemplary embodiments of apparatus, device and method which can be attached to a probe, including an endoscope. The exemplary apparatus and method can be utilized and/or performed in conjunction with any endoscope, as well as with laparascopic instruments and/or a resectoscope instrumentation.

### BACKGROUND INFORMATION

Endoscopic arrangements generally use a rigid or flexible endoscope with accessory channel(s) to aid diagnosis, carry out treatment or inspection. The endoscope can be inserted into an animal or human body in medical applications, including gastrointestinal tract, respiratory tract, and other internal organs, or other environments with special interests or under extreme conditions. The field of view of current endoscopic arrangements is generally limited by its configuration or light delivery and lens system, which only allows forward or another single directional field of view. The limitation of current endoscopic and laparascopic devices to provide only one field of view typically decreases the diagnostic accuracy, and can cause various lesions to be missed.

One method for visualizing the backwards field of view can include inserting a separate backwards-viewing endoscope through the accessory port of another endoscope. While this method provides backwards viewing, it can prevent the use of the accessory port while the backwards-viewing endoscope is inserted therein. The backwards-viewing endoscope would be withdrawn prior to biopsy acquisition, suction, and flushing, which can increase the time and cost of the procedure. A separate endoscope typically is furthermore sterilized following use, which can again increase the complexity of the procedure. The use of multiple endoscopes may also require multiple connections to an imaging console, which can further complicate the procedure. The additional complexity and cost of utilizing a separate endoscope can make it inconvenient to see in multiple directions, which may result in a deceased adoption of multiple endoscopes for obtaining images of additional fields of view.

Traditional methods for adhering endoscope accessories can include friction engagement, with or without the aid of gaskets, o-rings, or compressible adapter sleeves, and use of waterproof adhesive tape. Friction engagement has a drawback of being optimal for only one endoscope outer diameter, limiting use to a small portion of the varied-sized endoscope market. Both friction engagement and adhesives have the drawback of accelerated wear on the exterior material of the endoscope, shortening the life of the endoscope.

There is therefore a need for addressing at least some of the issues and/or deficiencies identified above. To that end, it may be beneficial to provide an endoscopic or laparascopic imaging system configuration that can facilitate multiple fields of view, including side and backwards fields, and which attaches in a secure and removable manner to the endoscope or laparoscope without damage or restricted movement, articulation, or other performance thereof.

### SUMMARY OF THE EXEMPLARY EMBODIMENTS

According to an exemplary embodiment of the present disclosure, an endoscope accessory apparatus can be provided which can enhance the utility of endoscopic procedures by, for example, providing additional fields of view for diagnostic screening. For example, with the exemplary embodiments of the present disclosure, it is possible for the accessory devices to securely, flexibly, and reversibly adhere to an endoscope probe of arbitrary size and minimally impedes articulation of the endoscope probe. A temporary stabilization of accessory apparatus position and orientation can be provided by friction engagement applied and/or enhanced by deformation, torsion and/or other mechanical alterations of the apparatus housing. Flexible, semi-flexible, and rigid embodiments are described. Engagement and disengagement can be achieved with forces applied with the hands or by other mechanical configurations and/or methods.

According to an exemplary embodiment of the present disclosure, an endoscope accessory apparatus can be positioned and secured to the endoscope probe through the use of compressive force supplied by a deformable housing structure covering at least one portion of the apparatus. Optical, electrical, and communication elements can be embedded or otherwise included in a flexible housing material with deformable electrical connections (e.g. metal conductors with coiled or meandering paths) such that optical, electrical, and communication performance of the apparatus is robust in the presence of moderate housing deformation, compression, expansion, elongation, flexion, torsion, or shear forces. The flexible housing material can also have an elastic mechanical property such that when stretched the apparatus may be positioned on the endoscope probe and when released the housing applies a compressive radial force against the probe. This compression can enhance a friction engagement of the exemplary apparatus to the endoscope probe, and can provide secure and stable positioning and favorable alignment of the apparatus with the endoscope probe.

In additional exemplary embodiments of the device and/or apparatus according to the exemplary embodiment of the present disclosure, the flexible materials can be comprised of any number of elastomers, including, e.g., room temperature vulcanizing silicone (RTV), polyurethane, or rubber.

In a further exemplary embodiment of the present disclosure, the flexible housing material can include a torsion element coiled along the inner diameter configured such that applying a torque to the device at each end of the torsion element, in a specific manner, increases the inner diameter of the housing such that the apparatus may be positioned around the endoscope probe. When the torque is released, the exemplary device/apparatus reduces the inner diameter and fixation is achieved through a radial compressive force and friction engagement. In yet a further exemplary embodiment of the device/apparatus according to the present disclosure, the torsion deformation between each end of the housing required to engage and/or disengage the holding friction is between about 5 and 90 degrees.

According to still another exemplary embodiment of the present disclosure, the housing can be comprised of rigid segments as well as flexible segments. One or more illumination sources, optical imaging elements and optical detectors can be contained in the rigid segment(s) to maintain preferred arrangement thereof, while electrical and battery components may be embedded in flexible segments. In such exemplary embodiment, the inner diameter of the rigid segments can be larger than the outer diameter of the probe. In addition or alternatively, the inner diameter of the rigid segments of the device housing can be between about 0.050 mm and 0.150 mm, e.g., larger than the outer diameter of the exemplary probe.

In another exemplary embodiment of the present disclosure, one or more outer surfaces of the rigid segments of the housing can be made of textured material to facilitate gripping with gloved fingers. In another embodiment of the device shallow scalloped indentations are provided to facilitate gripping with gloved fingers.

According to yet further exemplary embodiment of the present disclosure, the optical, electrical, communication and/or power storage components can be provided inside a flexible housing material that is configured with a bi-stable property. In a first exemplary stable state, the housing material can extend and be configured as a long rigid strip. The strip can be further configured against the outer surface of an endoscope probe. Upon a mechanical flexion, the housing can then quickly conform to a further stable state where the strip is configured as a coiled strip wrapping around and constricting around the endoscope probe. The housing can have residual flexibility in the second stable state such that the endoscope probe can be allowed to freely articulate while maintaining tight contact with the apparatus. To remove the exemplary apparatus from the endoscope probe, the housing material can be manually unwound to return the first stable state.

In still another exemplary embodiment of the present disclosure, a strain-sensitive electrical element can be provided in the flexible housing material and configured such that when the device is in the resting, non-expanded state the electrical components of the device are inactivated. The strain-sensitive electrical element can be further configured such that when the device is in the expanded state and/or positioned on the probe the electrical components of the device are activated.

According to yet another exemplary embodiment of the present disclosure, a computer arrangement can be provided to count the number of times the apparatus has been activated. The computer arrangement can be further configured to deactivate and/or render the apparatus inoperable once a predetermined number of uses has been reached.

In a further exemplary embodiment of the present disclosure, the housing can include a number of hinged retention arrangements that can be opened by a compressive force applied to the outer housing of the device. A spring loading arrangement can be provided and configured such that when the compressive force is removed, the hinged retention arrangements apply a radial compressive force against the probe to prevent a translation and/or a rotation of the device with respect to the probe.

According to still a further exemplary embodiment of the present disclosure, the outer housing can be comprised of a flexible material with a seam that extends along the axial length of the housing. The seam can be destroyed to facilitate the removal of the apparatus from the endoscope probe. In this embodiment the apparatus is disposable and designed for single use. A disengagement of the seam can also act to destroy at least one portion of the electrical system to ensure the apparatus cannot be reused.

For example, a probe coupling apparatus can be provided according to an exemplary embodiment of the present disclosure which can include, e.g., at least two opposing edges, one of the edges being configured to receive a tip of a probe. The exemplary apparatus can also include an orifice provided between the edges and a portion provided between the opposing edges and extending along the orifice. The portion can have a structure such that, when a force is applied to a section of the portion, a cross-sectional area of the orifice is increase, and, when the force is released, the area is decreased.

In an exemplary configuration, the further portion can include a spring, which can be a helical spring that extends along a direction between the edges. The helical spring can have a central portion that expands when force is applied, and decreased when the force is released. The force can be generated or increased by rotating at least one of the edges in a first direction. The force can be decreased or removed when the one of the edges is rotated in a second direction which is opposite to the first direction. In addition or alternatively, the helical spring can have a central portion and a distal portion. The central portion can have a diameter that is smaller than a diameter of the distal portion.

In another exemplary configuration, the exemplary probe coupling apparatus can include an outer section which encloses the orifice and the portion. For example, at least one area of the outer section can be more flexible than a further area of at least one of the edges.

According to still another exemplary configuration, at least one of the edges can have a bore with a first cross-sectional area, and the portion (provided between the opposing edges) can have a second cross-sectional area which is smaller than the first cross-sectional area when the force is not applied. The second cross-sectional area can be larger than the first cross-sectional area when the force is applied.

In another exemplary configuration, when a tip of a probe is inserted into the orifice and the force is release, the portion (provided between the opposing edges) can be coupled to the tip in a frictionally-maintaining manner, and can prevent a motion of the tip with respect to the portion. For example, when the force is reapplied, the portion can be at least partially decoupled from the tip so as to allow the tip to be removed from the orifice.

In addition or alternatively, the portion can include at least one lever and at least one spring, and the level can have a first end provided away from the orifice and a second end provided closer to the orifice and connected to the spring. For example, when the force is applied to the first end, the second end can cause a compression of the spring so as to increase a cross-sectional area of the portion. The compression of the spring can be provided in a direction that is approximately orthogonal to a direction of extension of the orifice.

According to a further exemplary configuration, the portion (provided between the opposing edges) can include at least one leaf spring and tooth-shaped sections which extend from the edges. The leaf spring(s) can have a first end fixed to one of the tooth-shaped sections and a second end fixed to another one of the tooth-shaped sections, and a middle portion which extends into the orifice. The application of the force can cause the deformation of the leaf spring(s) such that the middle portion is moved away from the orifice.

These and other objects, features, and advantages of the exemplary embodiments of the present disclosure can become apparent upon reading the following detailed description of the exemplary embodiments of the present disclosure, when taken in conjunction with the appended claims.

### BRIEF DESCRIPTION OF THE DRAWINGS

Further objects, features, and advantages of the present disclosure can become apparent from the following detailed description taken in conjunction with the accompanying Figures showing illustrative embodiments of the present disclosure, in which:

FIG. 1A-1F are front and side-views of an exemplary apparatus using a helical torsion spring and power switch configuration according to an exemplary embodiment of the present disclosure;

FIG. 2 is a three-dimensional elevational illustration of the exemplary apparatus of FIGS. 1A-1F using the helical torsion spring;

FIG. 3A-3B are side views of the exemplary apparatus using spring-loaded retention hinge pins according to another exemplary embodiment of the present disclosure;

FIG. 4A-4C are isometric and front views of the exemplary apparatus using a configuration of expandable linear springs according to still another exemplary embodiment of the present disclosure;

FIG. 5A-5C are isometric views of an exemplary apparatus using a tearable seam according to a further exemplary embodiment of the present disclosure; and

FIGS. 6A and 6B are isometric views of an exemplary apparatus using a bi-stable housing structure according to an exemplary embodiment of the present disclosure.

Throughout the drawings, the same reference numerals and characters, unless otherwise stated, are used to denote like features, elements, components, or portions of the illustrated embodiments. Moreover, while the present disclosure will now be described in detail with references to the figures, it is done so in connection with the illustrative embodiments and is not limited by the particular embodiments illustrated in the figures, or the appended claims.

### DETAILED DESCRIPTION OF EXEMPLARY EMBODIMENTS

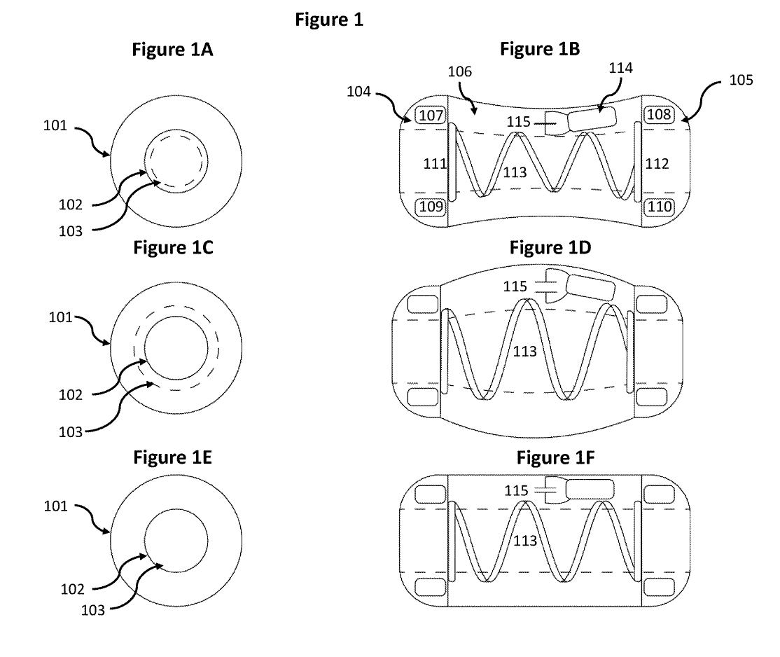

Using the exemplary embodiments of the apparatus, system, and method of the present disclosure, it is possible to manipulate a housing structure of an endoscope accessory apparatus to provide stable and reversible fixation to and alignment with an endoscope probe. For example, FIG. 1A-1F illustrate front and side-views of an exemplary apparatus using a helical torsion spring and power switch configuration according to an exemplary embodiment of the present disclosure. In this exemplary embodiment, a housing is provided that comprises two (or more) rigid ring segments connected by a flexible cylindrical segment.

FIG. 1A shows a front view illustrating a rigid segment outer diameter (101, solid line) and inner diameter (102, solid line) and a flexible segment resting inner diameter (103, dashed line). The exemplary housing can be be configured to adhere to an endoscope probe with outer diameter greater than the resting inner diameter (103) but less than the rigid ring segment inner diameter (102). FIG. 1B shows a side view of such exemplary embodiment with solid ring segments (104 and 105), and flexible housing segment (106). The rigid elements may contain optical and/or electrical components (107-110) within the ring, while leaving clear cylindrical openings (111-112) to allow the passage of the endoscope probe. The flexible segment can contain a helical spring component (113) that connects the rigid segments together. Electrical elements, computer arrangements, and/or power storage components (e.g. batteries, 114) may be positioned in the flexible segments. These electrical components may further contain a strain-sensing element (115) that is configured to activate and/or deactivate the electronic and/or computer arrangement.

FIG. 1C shows a side view of the exemplary apparatus in an expanded state. Gripping the two rigid ring elements (using, e.g. hands or other mechanical means) and applying a torque causes the spring element to expand. This expansion causes an increase in inner diameter of the flexible segment (103). FIG. 1D shows a side view of the exemplary apparatus in the expanded state. For example, the spring element (113) causes an expansion of the flexible member and also an activation of the strain-sensitive element 115. This exemplary activation of the strain-sensitive element then causes the electrical elements to activate or de-activate. In the expanded state, the apparatus may be positioned around the endoscope probe in a favorable configuration alignment. Once in position, the torque may be removed from the rigid segments causing the spring element (113) to relax and apply a compressive force onto the endoscope probe.

FIG. 1E shows a front view of the exemplary embodiment of the apparatus with inner diameter of the flexible segment (103) matching the outer diameter of the endoscope probe.

FIG. 1F shows a side-view of the exemplary apparatus when positioned onto the endoscope probe. For example, the strain-sensitive element (115) can be configured to automatically activate the electrical components in this position.

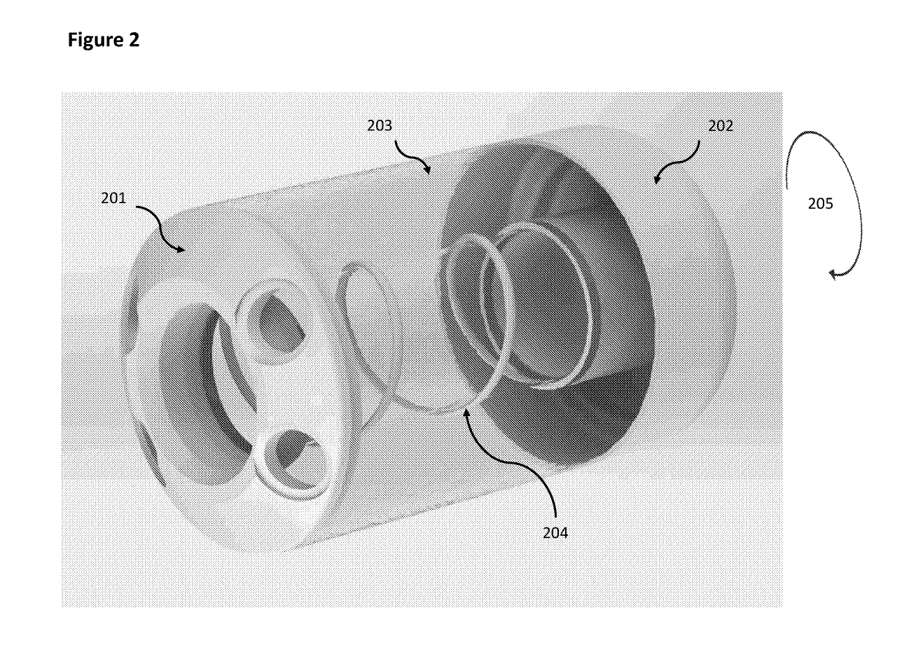

FIG. 2 shows a three-dimensional drawing of the exemplary apparatus shown in FIGS. 1A-1F using the helical torsion spring. For example, rigid segments (201, 202) can be configured to house optical and/or electrical components that are sensitive to deformation and/or strain. A flexible segment (203) can be provided for connecting the rigid segments. A torsion spring (204) can be provided within the flexible housing segment (203) that connects to the outer rigid segments (201, 202). The spring element can be configured to temporarily expand or contract by holding the front rigid segment (201) stationary and rotating the back rigid segment (202) in a torsion direction (205).

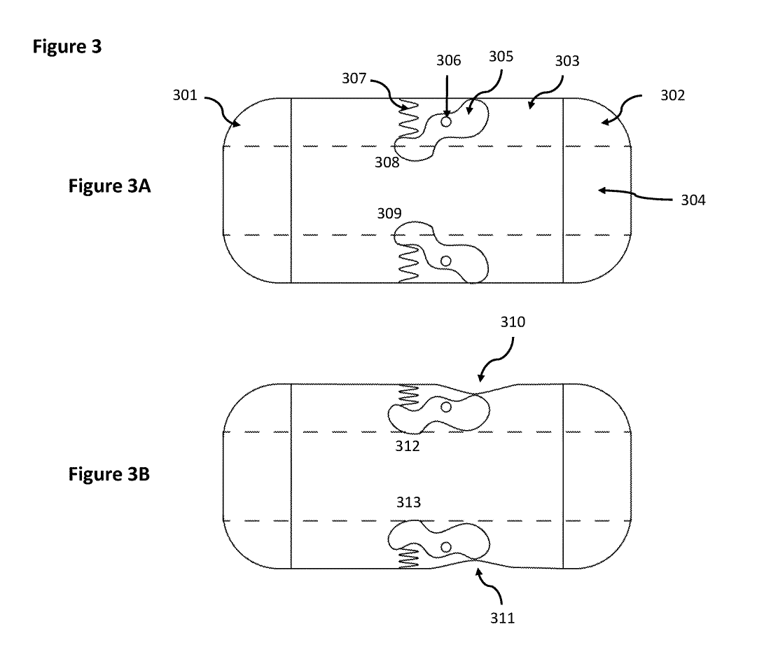

In another embodiment of the present disclosure, a rigid housing can be provided with local flexible segments for engagement and disengagement via compressive force. For example, FIGS. 3A and 3B illustrate side views of an exemplary apparatus using spring-loaded retention hinge pins.

In particular, FIG. 3A shows the exemplary embodiment of the apparatus with outer housing segments (301, 302) connected by a rigid inner segment (303). These exemplary housing components can be provided with an inner cylindrical channel (304) that allows the free (or substantially free) passage of an endoscope therein. The exemplary apparatus can be further provided with two or more hinge pin elements (305) configured to rotate about a fixed point (306), and held in a resting position via spring elements (307) provided within the housing. Some aspects and/or portions of the hinge can be configured to press or extend into the inner channel (304) to prevent the free passage of an endoscope probe.

FIG. 3B shows a side view of the exemplary embodiment of the present disclosure in an engaged stated. For example, the localized exterior regions (310, 311) of the rigid housing segment (303) can be configured and/or structured to be flexible, and allow for a compression (by fingers or other mechanical means) that causes the spring elements (307) to compress, and the blocking aspects or portions (312, 313) to recede into the housing segment (303) and allow the free passage of the endoscope probe. When the exemplary apparatus is configured in a specific position and alignment on the endoscope probe, the exterior regions and/or portions (310, 311) can be released causing the hinge pins to engage the endoscope probe and stabilize the apparatus.

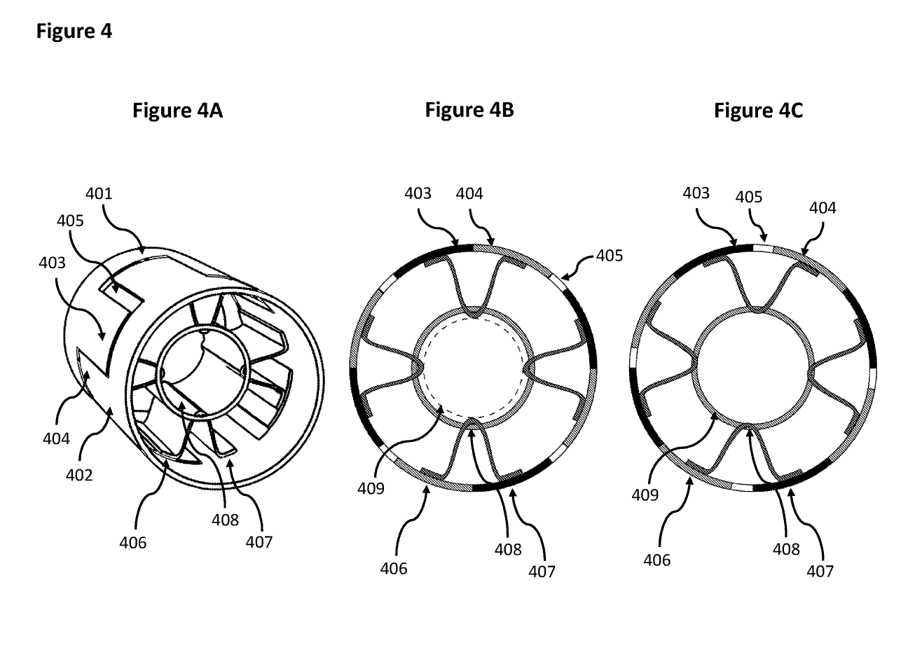

In another exemplary embodiment of the present disclosure, a two-part interdigitated housing may be provided to allow for engagement and disengagement via an applied torque. For example, FIG. 4A shows the exemplary two-element housing structure provided with two cylindrical segments (401, 402) having interlocking members (403, 404). The interlocking members can be configured or designed such that at least one gap (405) allows the free rotation of one segment with respect to the other over a finite rotational range (e.g., between 5 and 90 degrees). The cylindrical segments (401, 402) can be attached to each other through at least one linear spring with attachment points (406-407). The peak of the linear spring can be further configured to compress a flexible inner housing segment (408) in the resting state.

FIG. 4B shows a front cross-sectional view of the exemplary embodiment of the apparatus of FIG. 4A, with, for example, four front segments (403), four back segments (404), and four gaps (405). It should be understood that other numbers of the front segments (403), (back segments (404) and gaps (405) can be used effectively within the definition and scope of the present disclosure. The front and back segments (403, 404) can be attached to one another by an arrangement of four linear springs with attachments points (406, 407) and compression points (408). The compressed flexible inner segment can restrict a free movement within the exemplary apparatus to objects with maximum outer diameter (409). In one exemplary embodiment of the present disclosure, this maximum outer diameter can be smaller than the outer diameter of an endoscope probe.

FIG. 4C shows a front cross-sectional view of the exemplary embodiment of the apparatus of FIG. 4A, in an exemplary engagement. The exemplary engagement can be caused by, e.g., holding the front segments (403) stationary, and applying a torque to the back segment (404). The movement of the segments (403, 404) can cause the compression points of the linear springs (408) to enlarge the maximum outer diameter (409), and allow a free (or substantially free) movement of the endoscope probe. Once the apparatus is configured in a particular position and alignment on the endoscope probe, the torque can be released, and the apparatus can stabilize.

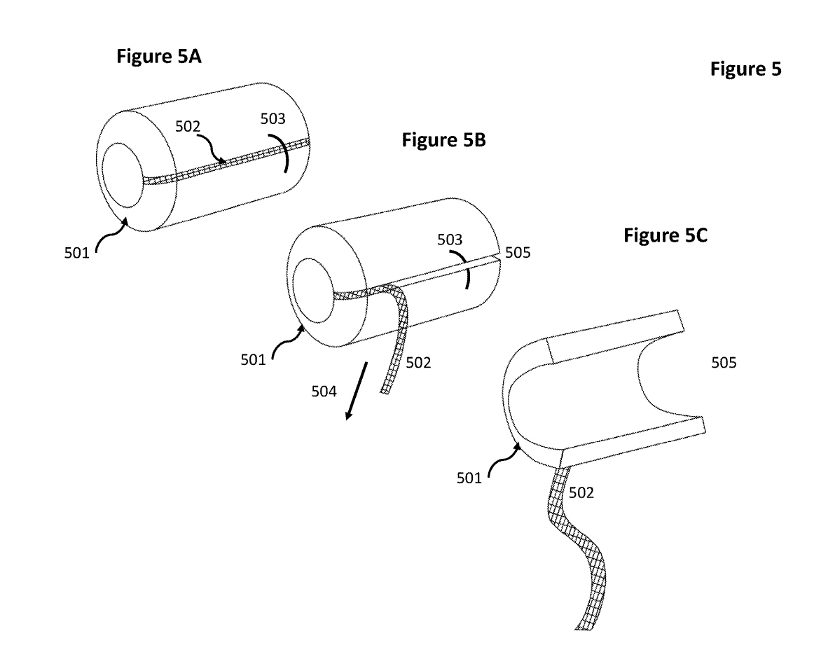

In yet another exemplary embodiment according the present disclosure, as shown in FIG. 5A, an endoscope accessory apparatus (501) can be provided with a removable seam (502) that can hold the exemplary apparatus together. The exemplary apparatus can be further configured to provide at least one wire (503) that can be integrated into the seam. As shown in FIG. 5B, the seam (502) can be disengaged from the exemplary apparatus (501) by pulling away from the housing in a particular direction (504). Once the seam is removed, an opening in the housing (505) can created through which the endoscope probe can be removed. In one exemplary embodiment, the disengagement of the seam can also destroy the electrical connections (503) that can be important for a functionality of one exemplary embodiment of the apparatus. This exemplary feature of the exemplary apparatus can prevent the apparatus from being re-used in multiple procedures. As shown in FIG. 5C, the seam can be configured to remain attached to one surface of the housing while allowing the opening (505) to fully detach the exemplary apparatus from the endoscope probe.

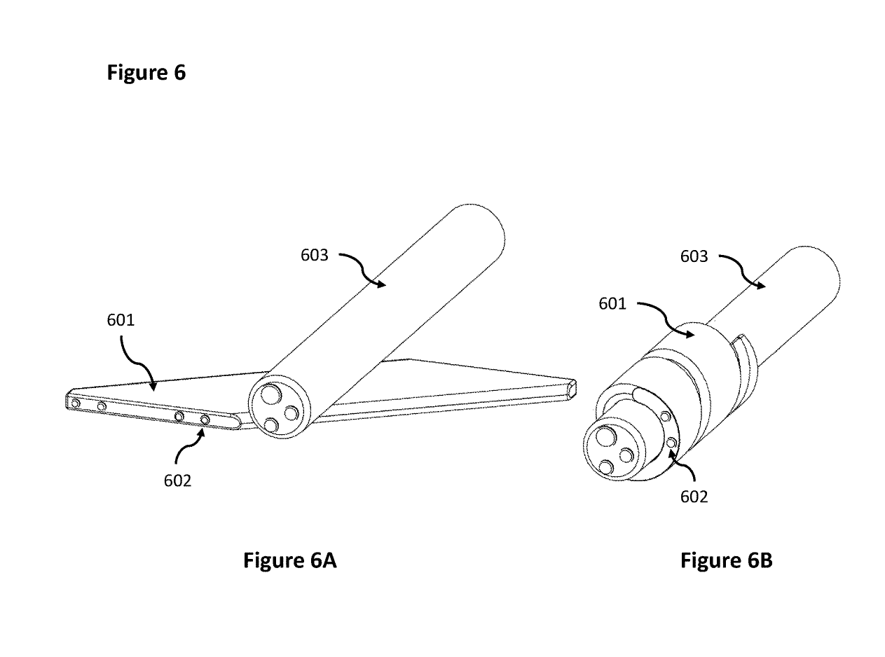

According to yet another exemplary embodiment of the present disclosure, the endoscope accessory apparatus (601) can be provided with a flexible bi-stable housing structure for engagement and disengagement via conformal changes. As shown in FIG. 6A, the endoscope accessory apparatus (601) can be provided with a flexible bi-stable housing element that conforms to one of two stable physical states. A first exemplary open state can approximate a long, flat ribbon and provides at least one optical window (602) on at least one surface. An endoscope probe (603) can be positioned and aligned with the apparatus in the open state. Gentle wrapping of the housing apparatus around the endoscope probe can cause a rapid configuration change to a second closed state, as shown in FIG. 6B. The closed state applies compressive force to the endoscope probe and enhances friction engagement to secure the apparatus position and alignment. Residual flexibility of the housing in the closed state may facilitate the endoscope probe to articulate while retaining the preferred apparatus alignment.

All of the exemplary embodiments of the present disclosure described herein can be suitable for fixation of a device that facilitates additional fields of view during a colonoscopic, baroscopic, laparascopic, angioscopic, or other endoscopic procedure. Exemplary embodiments of device/apparatus providing the multidirectional fields of view can be attached to the endoscope, which does not generally affect the normal function of the endoscope, such as, e.g., the accessory port and angulations. The exemplary apparatus can provide continuous and simultaneous forward and/or multidirectional views during colonoscopic, baroscopic, laparascopic, angioscopic and/or other endoscopic procedures. Exemplary devices can be affixed to the endoscope according to the exemplary embodiments described herein, and applied to rigid, flexible, wireless and/or telescoping endoscope to provide, e.g., continuous multidirectional views of animate and inanimate hollow spaces. The exemplary dimensions of the exemplary apparatus may be scaled to fit specific scope sizes.

Exemplary embodiments of the present disclosure can relate generally to exemplary configuration of optical and electronic elements, and to the application(s) thereof in exemplary endoscopic imaging systems which can be used with medical and industrial applications to improve the field of view, speed and efficiency of an endoscopic procedure.

According to another exemplary embodiment of the present disclosure, the exemplary device/apparatus can include a video/analog/digital image sensor/camera and/or signal detectors and sensors that can be embedded in a cap, and which can be attached to the part of the endoscope. In a further exemplary embodiment of the present disclosure, multiple configurations of signals and/or images sensors/detectors can be contained within the cap.

According to yet another exemplary embodiment of the present disclosure, the signals and/or images can be transmitted remotely via a wireless transmitter. In addition or as an alternative, a battery source can be contained within the cap that can power the signals and/or images sensors/detectors and illumination sources without requiring an external connection.

The foregoing merely illustrates the principles of the disclosure. Various modifications and alterations to the described embodiments can be apparent to those skilled in the art in view of the teachings herein. The foregoing merely illustrates the principles of the disclosure. Various modifications and alterations to the described embodiments can be apparent to those skilled in the art in view of the teachings herein. Indeed, the arrangements, systems and methods according to the exemplary embodiments of the present disclosure can be used with and/or implement any endoscope and/or probe system, and for example with those described in U.S. Patent Application Ser. No. 61/618,225, filed Mar. 30, 2013, International Application PCT/US2013/031948, filed Mar. 15, 2013, U.S. Patent Application Ser. No. 61/856,152, filed Jul. 19, 2013; U.S. Patent Application Ser. No. 61/985,824, filed Apr. 29, 2014; and, International Application PCT/US2014/047034, filed Jul. 17, 2014, the disclosures of which are incorporated by reference herein in their entireties. It will thus be appreciated that those skilled in the art can be able to devise numerous systems, arrangements, and procedures which, although not explicitly shown or described herein, embody the principles of the disclosure and can be thus within the spirit and scope of the disclosure. In addition, all publications and references referred to above can be incorporated herein by reference in their entireties. It should be understood that the exemplary procedures described herein can be stored on any computer accessible medium, including a hard drive, RAM, ROM, removable disks, CD-ROM, memory sticks, etc., and executed by a processing arrangement and/or computing arrangement which can be and/or include a hardware processors, microprocessor, mini, macro, mainframe, etc., including a plurality and/or combination thereof. In addition, certain terms used in the present disclosure, including the specification, drawings and claims thereof, can be used synonymously in certain instances, including, but not limited to, e.g., data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it can be explicitly being incorporated herein in its entirety. All publications referenced above can be incorporated herein by reference in their entireties.

What is claimed is:

1. A probe coupling apparatus, comprising:
at least two opposing edges, one of the edges being configured to receive a tip of a probe;
an orifice provided between the edges; and
a portion provided between the opposing edges and extending along the orifice, wherein the portion has a structure such that, when a force is applied to a section of the portion, a cross-sectional area of the orifice is increase, and wherein, when the force is released, the area is decreased.

2. The probe coupling apparatus according to claim 1, wherein the further portion includes a spring.

3. The probe coupling apparatus according to claim 2, wherein the spring is a helical spring that extends along a direction between the edges.

4. The probe coupling apparatus according to claim 3, wherein the helical spring has a central portion that expands when force is applied, and decreased when the force is released.

5. The probe coupling apparatus according to claim 4, wherein the force is generated or increased by rotating at least one of the edges in a first direction, and wherein the force is decreased or removed when the one of the edges is rotated in a second direction which is opposite to the first direction.

6. The probe coupling apparatus according to claim 3, wherein the helical spring has a central portion and a distal portion, wherein the central portion has a diameter that is smaller than a diameter of the distal portion.

7. The probe coupling apparatus according to claim 1, further comprising an outer section which encloses the orifice and the portion, wherein at least one area of the outer section is more flexible than a further area of at least one of the edges.

8. The probe coupling apparatus according to claim 1, wherein at least one of the edges has a bore with a first cross-sectional area, and wherein the portion has a second cross-sectional area which is smaller than the first cross-sectional area when the force is not applied.

9. The probe coupling apparatus according to claim 8, wherein the second cross-sectional area is larger than the first cross-sectional area when the force is applied.

10. The probe coupling apparatus according to claim 1, wherein, when a tip of a probe is inserted into the orifice and the force is release, the portion is coupled to the tip in a frictionally-maintaining manner, and prevents a motion of the tip with respect to the portion.

11. The probe coupling apparatus according to claim 1, wherein, when the force is reapplied, the portion is at least partially decoupled from the tip so as to allow the tip to be removed from the orifice.

12. The probe coupling apparatus according to claim 1, wherein the portion includes at least one lever and at least one spring, and wherein the level has a first end provided away from the orifice and a second end provided closer to the orifice and connected to the spring.

13. The probe coupling apparatus according to claim 12, wherein, when the force is applied to the first end, the second end causes a compression of the spring so as to increase a cross-sectional area of the portion.

14. The probe coupling apparatus according to claim 13, wherein the compression of the spring is provided in a direction that is approximately orthogonal to a direction of extension of the orifice.

15. The probe coupling apparatus according to claim 1, wherein the portion includes at least one leaf spring and tooth-shaped sections which extend from the edges.

16. The probe coupling apparatus according to claim 15, wherein the at least one leaf spring has a first end fixed to one of the tooth-shaped sections and a second end fixed to another one of the tooth-shaped sections, and a middle portion which extends into the orifice.

17. The probe coupling apparatus according to claim 16, wherein the application of the force causes the deformation of the at least one leaf spring such that the middle portion is moved away from the orifice.
